(19) United States
(12) Patent Application Publication
(10) Pub. No.: US 2007/0272279 A1
(43) Pub. Date: Nov. 29, 2007

(54) Sanitizing system

(76) Inventor: Robert D. Foster, Fort Worth, TX (US)

Correspondence Address: LYNN E BARBER, P O BOX 16528, FORT WORTH, TX, 76162, US

(21) Appl. No.: 11/440,545

(22) Filed: May 25, 2006

(51) Int. Cl. B08B 1/02; B08B 3/00

(52) U.S. Cl. 134/32; 134/198; 134/34; 134/137

(57) ABSTRACT

A system and method for sanitizing articles including a rectangular apparatus defining an enclosure through which the articles may be transported, a tank for sanitizing solution, sprayers for spraying sanitizing solution on the top and bottom of articles in the enclosure, and a container for catching used sanitizing solution; a mechanism for moving the articles through the rectangular enclosure from the first entry end and out of the enclosure at the second exit end; a mechanism for automatic triggering of release of water from the tank when an article enters the enclosure; a mechanism for carrying used water to either the container for sanitizing solution or a drain system; and a sanitizing solution capable of disinfecting the articles when sprayed on the articles without requiring rinsing of the articles after the articles have been sprayed with the sanitizing solution.

### BACKGROUND OF THE INVENTION

1. Field of the Invention

This invention relates to a system for sanitizing objects, and in particular relates to a method and apparatus for sanitizing items such as shopping carts.

2. Description of the Related Art

Carts used by shoppers in grocery stores, discount stores and other types of stores to transport items that the shopper is going to purchase are variously known as shopping carts, grocery carts, delivery carts or trolleys. These carts are used over and over, by many people each day. The shoppers using these carts may have a communicable illness or have hands that are dirty or laden with infectious agents such as bacteria and viruses. Examples of infectious agents that may be spread in this way are fecal coliforms, cold and influenza viruses, and food-borne or skin-borne pathogens such as Salmonella and Staphylococcus. For carts that have a child-seat, a child sitting in the seat may be shedding infectious agents by sneezing, drooling and the like. The carts are also often used to hold items that are themselves dirty or contaminated by their very nature, such as plants in soil, and leaking packages of poultry or other meat, or the items may have been contaminated prior to being placed in the cart, such as by being stored under unsanitary conditions. Also, most stores that have carts allow the shoppers to wheel the carts outside to deliver the purchased items to their cars. Often the carts are then left outside, where they may be exposed to bird droppings or other unsanitary conditions.

These situations become particularly problematic when there are disease outbreaks or epidemics, or there are particularly contagious diseases around or animal diseases, such as bird flu, which have become transmittable to humans. A related problem to that presented by shopping carts is the re-use of carts in hospital or other medical environments.

Most shopping cart manufacturers recommend periodic washing of their carts, e.g., quarterly, often primarily to remove environmental contaminates, including particulates that might corrode the carts or otherwise be a problem. The traditional method of dealing with obviously dirtied shopping carts has been to hire someone to wash the carts, for example, with a high-pressure water wash.

As the realization of the hazards presented by unclean shopping carts has increased, however, a variety of washing systems have been developed. Many early methods of cleaning carts concentrated on cleaning the handles, where most contact with the shoppers occurs, but neglected to clean the body of the cart where various items may have leaked. Others concentrated on cleaning cart wheels (e.g., U.S. Pat. No. 5,454,391).

Numerous methods of cleaning the entire cart have been developed. An early example is the mobile cart washer of Thornton (U.S. Pat. No. 3,444,867) in which a ramped wash station has nozzles mounted to spray a heated wash fluid at the carts, held at their lower front by fingers on a continuous, moving conveyor chain. The cleaning apparatus of Pulliam (U.S. Pat. No. 4,279,263) has a wheeled base, an interior tank of cleaning liquid (water), a heater, a pump to pump the water to a nozzle and another pump to pump used water from a waste pan through a filter and into a storage tank.

The high pressure hot water cleaning system for grocery carts of Poitevin (U.S. Pat. No. 4,807,319) is a fully enclosed system in a trailer including a conveyor, rotating brushes, a spent fluid holding tank, a cleaning fluid supply tank, a sanitizing fluid supply tank, and a hand-operated wand to allow the operator to remove difficult accumulations from the carts. The apparatus for washing objects, such as shopping carts, of Luongo (U.S. Pat. No. 5,622,196) uses a conveyor belt with exterior sections on each side of a housing frame. Conveyed objects are sprayed with a cleaning solution, then washed with a pair of high speed rotating sprayer arms mounted exteriorly and directing spray through cut outs in the side of the housing, and then rinsed before exiting the housing.

The washer of Morris (U.S. Pat. No. 6,427,707) has a conveyor to take the carts in either direction through an enclosure, a plurality of spray nozzles to direct liquid inside the enclosure, an air curtain delivery assembly at each end of the enclosure, and means for recycling the sprayed liquid back to the nozzle.

Building on the single conveyor system, the invention of Knowlton et al. (U.S. 2005/0121057) has multiple speed conveyor belts to assist in pulling shopping carts out of a nested line or putting them back into the nested line.

Some of the continuous washing structures for wheeled items utilize U-shaped pathways on which the items being washed are conveyed (e.g., U.S. Pat. No. 5,993,739 for wheelchairs, and U.S. Patent Application No. 2004/0159336 utilizing a mobile unit for shopping carts).

The patent of Brackman et al. (U.S. Pat. No. 6,090,218) utilizes a linear tunnel housing and at least one pipe manifold with nozzles positioned in the housing to provide at least one treatment selected from pre-wash, wash, disinfecting (e.g. hypochlorite), and rinse zones, and optional air-dry and heat zones, or may utilize a single zone with preprogrammed treatments. There may be batch treatments (no movement) or continuous movement treatments.

The shopping cart wash tunnel of Mueller (U.S. Patent Application 2006/0011220) utilizes a conveyor, or other means known in the art, to move the carts into the apparatus. Sanitizing agents (e.g., soap and water, chemical agents, or a combination of disinfecting fluids) are automatically dispensed onto the carts. There is a means for detecting the position of the shopping arts in the tunnel and an array of nozzles for spraying when the carts are conveyed under them. Alternatively, the carts in this invention may remain stationary and a spray gantry may move within the tunnel and travel over the carts. In either case, the treatments may be programmed into the system. Waste fluids are diverted from the system to a waste reclamation unit, and may be recycled or otherwise properly disposed of.

The shopping cart sanitization system of Holbrook (U.S. Patent Application 2005/0217701) also provides an enclosure with nozzles that may be positioned anywhere within the enclosure to provide disinfecting to any part of the cart. Fluid may be emitted in a spray or mist or stream as the cart is conveyed through the enclosure. A sensor is provided to indicate the cart position.

Numerous other mechanisms to wash carts and related objects have been developed, and include various oscillating sprayers (U.S. Pat. No. 4,452,263), timed sprayers (U.S. Pat. No. 4,739,779), and different conveying mechanisms for cleaning carts and cars (e.g., U.S. Pat. No. 4,462,315).

Thus, the prior systems for cleaning of carts typically utilize multiple treatments, and if a non-water disinfectant is used, a rinse is typically used. Many of the systems are not portable, may require connection to a source of water, may require a permanent or substantial installation on the premises where the system is used, or are very expensive or difficult to construct.

It is therefore an object of the invention to provide a portable self-contained system for sanitizing items such as shopping carts. Other objects and advantages will be more fully apparent from the following disclosure and appended claims.

### SUMMARY OF THE INVENTION

The invention herein is a system, apparatus and method for sanitizing items such as shopping carts. The invention utilizes a rectangular apparatus defining an enclosure through which the articles may be transported, a sanitizing solution capable of disinfecting the articles when sprayed on the articles without requiring rinsing of the articles after the articles have been sprayed with the sanitizing solution; a tank for the sanitizing solution, sprayers for spraying sanitizing solution on the top and bottom of articles in the enclosure; a container for catching used sanitizing solution; a mechanism for moving the articles through the rectangular enclosure from the first entry end and out of the enclosure at the second exit end; a mechanism for automatic triggering of release of water from the tank when an article enters the enclosure; a mechanism for carrying used sanitizing solution to sprayers for washing to lower areas of the articles, and a mechanism for carrying excess used sanitizing solution to a drain system.

Other objects and features of the inventions will be more fully apparent from the following disclosure and appended claims.

### BRIEF DESCRIPTION OF THE DRAWINGS

FIG. 1A is a schematic upper view of the version of the invention in which no guide rails are used, showing carts positioned at the entry end of the invention. FIG. 1B is a schematic upper view of the invention in which guide rails are used.

FIG. 2 is a cut-away perspective side view of the apparatus of the invention.

FIG. 3 is a perspective side view of a sheet used to make the sides of the invention.

FIG. 4 is a perspective view of a pan used at the bottom of the invention.

FIG. 5 is a perspective view of a leg of the invention.

FIG. 6 is partial perspective view of an assembled side and leg of the invention.

FIG. 7 is a perspective end view of the entry end of the apparatus of the invention showing the rails.

FIG. 8 is a perspective view of an upper panel of the invention.

FIG. 9 is an elevational view of the carpet that is mounted at each end of the invention.

FIG. 10 is a perspective view of the cover pieces of the invention.

FIG. 11 is a perspective view of the top divider of the invention.

FIG. 12 is a perspective view of a side rail of the invention.

FIG. 13 is a perspective view of an equipment support bracket for use at the end of the apparatus of the invention.

FIG. 14 is a perspective view of an equipment support bracket for use within the apparatus of the invention.

FIG. 15 is a perspective view of the rail system of the invention.

FIG. 16 is a perspective view of the external triangular guide rail of the invention.

FIG. 17 is a perspective view of one of the hat sections that may be used in the invention.

FIG. 18 is a partial perspective view of the apparatus showing mounted equipment support brackets.

FIG. 19 is a perspective view of the apparatus of the invention showing the preferred sprayer locations.

FIG. 20 is an elevational view of a drain pit that may be used with the invention.

### DETAILED DESCRIPTION OF THE INVENTION AND PREFERRED EMBODIMENTS THEREOF

The present invention provides a system, apparatus and method for sanitizing items such as shopping carts. In summary, the invention utilizes a rectangular apparatus defining an enclosure through which the articles may be transported, a tank for sanitizing solution, sprayers for spraying sanitizing solution on the top and bottom of articles in the enclosure, and a container for catching used sanitizing solution; a mechanism for moving the articles through the rectangular enclosure from the first entry end and out of the enclosure at the second exit end; a mechanism for automatic triggering of release of water from the tank when an article enters the enclosure; a mechanism for carrying used water to either the container for sanitizing solution or a drain system; and a sanitizing solution capable of disinfecting the articles when sprayed on the articles without requiring rinsing of the articles after the articles have been sprayed with the sanitizing solution.

For use with shopping carts, the sanitizing apparatus of the invention is preferably placed in the parking lot adjacent to the store where the shopping carts are used. Carts located in the parking lot are moved to the back of the sanitizing apparatus for cleaning. The carts are either hooked to a pull strap and a winch located at the front of the machine pulls the cart through, or the carts may be pushed through by hand. Inside the apparatus are a washer and sanitizer. A cart placed in the apparatus passes through three fogger nozzles where a mild solution of disinfectant, detergent and de-foamer is sprayed on the cart. At the bottom of the cart is a washer and sanitizer with recycled chemical from the front three spray nozzles. The cart then goes through a blower and exits the machine, ready for use. Because of the choice of chemicals used in the apparatus, no rinsing is necessary.

The preferred disinfectant chemical used in the apparatus of the invention is non-toxic to humans, preferably a quaternary ammonium chloride. This chemical exhibits effective disinfective properties against a wide variety of bacteria including Pseudomonas aeruginosa, Escherichia coli, Klebsiella pneumonia, Salmonella (schuttmuelleri and choleraesuis), Streptococcus (salivarius and faecalis) Brevibacterium (ammonia genes), Shigella dysenteries, Staphylococcus aureus, and Enterobacter aerogenes. The preferred chemical also has fungicidal (e.g., against pathogenic fungi, Trichophyton mentagrophyles) and virucidal properties against, for example, such viruses as influenza A2-Asian, Herpes simplex, Adenovirus type 5 and Vaccinia virus. These bactericidal, fungicidal and virucidal properties are present even in the presence of other materials such as soil or blood.

It is also important that this chemical does not leave a harmful residue on the carts when used according to the invention herein. Thus, the preferred chemicals are approved for use in food establishments, such as on previously cleaned food equipment or food contact items, without requiring a potable water rinse (e.g., USDA Code D2, EPA Reg. no. 1839-155), and are currently used for household, hospital, dairy, restaurants and related cleaning.

As preferably used herein, the concentrated chemical is mixed with water, preferably at 200-800 ppm. A preferred formulation is as approved by EPA Reg. No. 47371-130, for example, Formulation HWS-128 of H&S Chemicals Division, Lonza Inc., Allendale N.J.), containing: 5.07% didecyl dimethyl ammonium chloride; 3.38% n-alkyl (C₁₄ 50%, C₁₂ 40%, C₁₆ 10%) dimethyl benzyl ammonium chloride, and 91.55% inert ingredients, such as fragrances and water. Comparable disinfectant substances that may be used in the invention herein may be obtained from Stepan Company (Northfield, Ill.), for example, BTC 2125® (n-alkyl dimethyl benzyl ammonium chlorides and n-alkyl dimethyl ethylbenzyl ammonium chlorides).

In the discussion herein of the preferred embodiments of the invention, specific dimensions are used to aid in understanding of how the invention is made in the preferred embodiments. Other dimensions may of course by used as indicated by the size of item to be cleaned by the apparatus and by other constraints for particular uses without departing from the spirit and scope of the invention herein. Also, the metal structural components are preferably made of aluminum, or alternatively, they may be made of stainless steel.

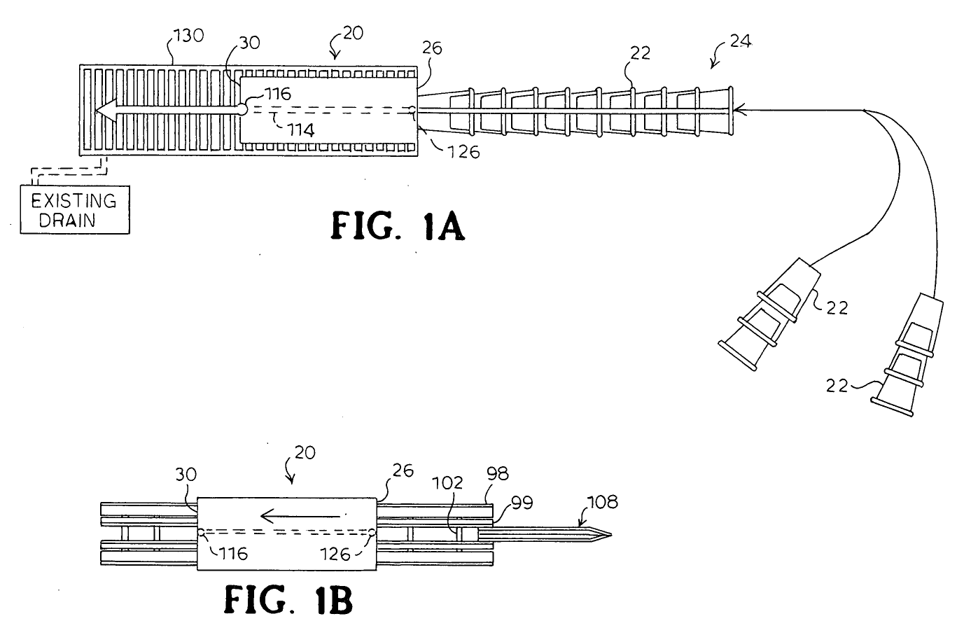

Referring in greater detail to the figures, FIG. 1A is a schematic upper view of the apparatus 20 of the invention (without guide rails) in use. FIG. 1B shows the apparatus 20 of the invention used with guide rails. In either version, carts 22 are moved from their various locations outside the apparatus 20, such as from individual and grouped locations in a store parking lot. The carts 22 are preferably nested together if the carts 22 are so designed, and the nested group 24 of carts 22 is moved into a first entry end 26 of the apparatus 20.

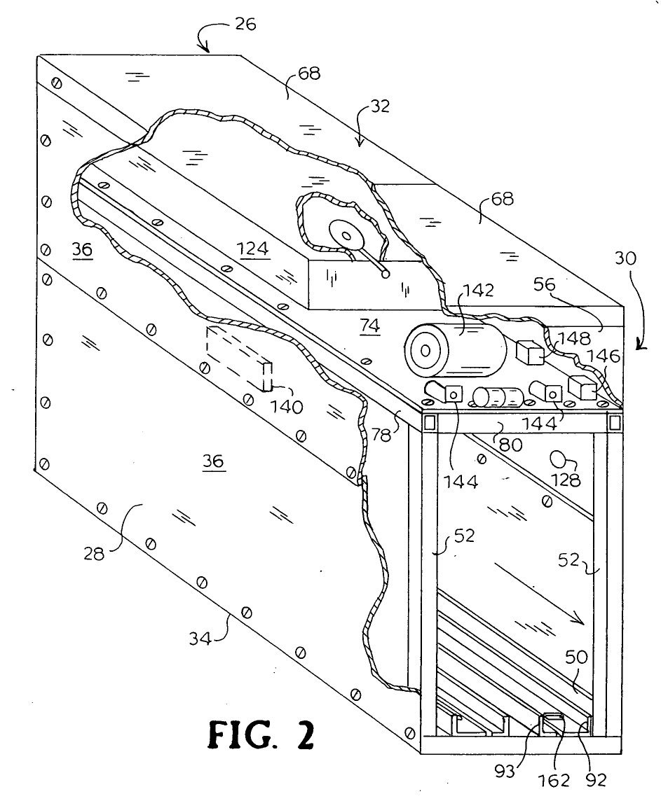

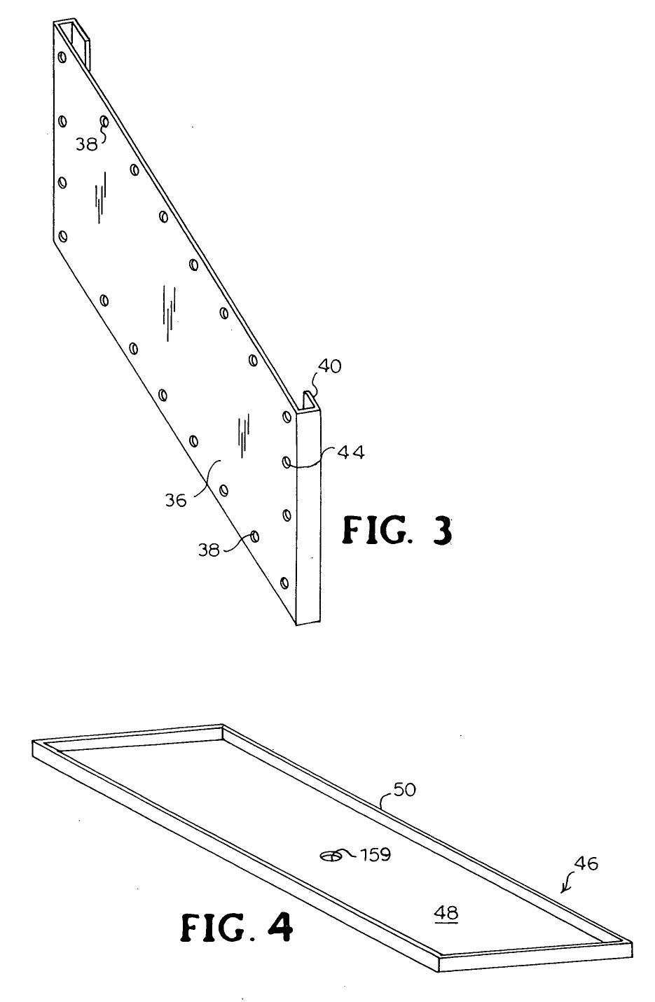

The apparatus 20 of the invention is basically a rectangular structure having sides 28, a first entry end 26, a second exit end 30, a top cover 32 and a bottom 34 (FIG. 2). The sides 28 of the rectangular structure (FIG. 3) are preferably about 72-84 inches high×about 91 inches long. Most preferably the sides 28 are 84×91 inches.

Each side 28 is preferably made of two stainless steel sheets 36 (0.063 inches thick) (FIG. 3) having a diamond pattern or other suitable pattern as is desired (not shown). Thus, for a standard 72-inch high apparatus 20, two side sheets 36, each measuring 36 inches high×91 inches long are used for each side. For a taller apparatus 20, a 36-high sheet and a 42-inch high sheet, or two 42-inch high sheets, or any other size as is desired, may be used for each side and assembled together in the same manner as for the apparatus 20 described herein. The two sheets 36 that are used for each side each have holes 38 (e.g., 0.187 inch diameter) at equal intervals (e.g., 5-inch intervals) along the top and bottom of each sheet 36. The two sheets 36 that make up each side 38 are fastened together by bolting through corresponding holes in the bottom of one sheet and top of the other sheet, or by other means known in the art. The vertical side edge of each sheet 36 is bent at a 90-degree angle to form a squared-U flange portion 40 as shown in FIG. 3 that has an interior width to accommodate the legs 52 (FIG. 3 and FIG. 6, discussed below)

The bottom 34 of the apparatus 20 comprises a pan 46 (FIG. 4), which in the preferred embodiment for standard shopping carts 22 has a flat bottom piece 48 that is 36.12 inches×91.187 inches, with three-inch high sides 50 welded to each other on the corners to form a pan that will hold used sanitizing solution. There is a central drain 159 as shown. Although not shown in FIG. 4, before forming pan 46 with sides 50 being welded together, bottom 48 is creased diagonally between each corner of the pan 46 and central drain 159, and then bottom 48 is again flattened, as is known in the art for strengthening a flat piece of metal.

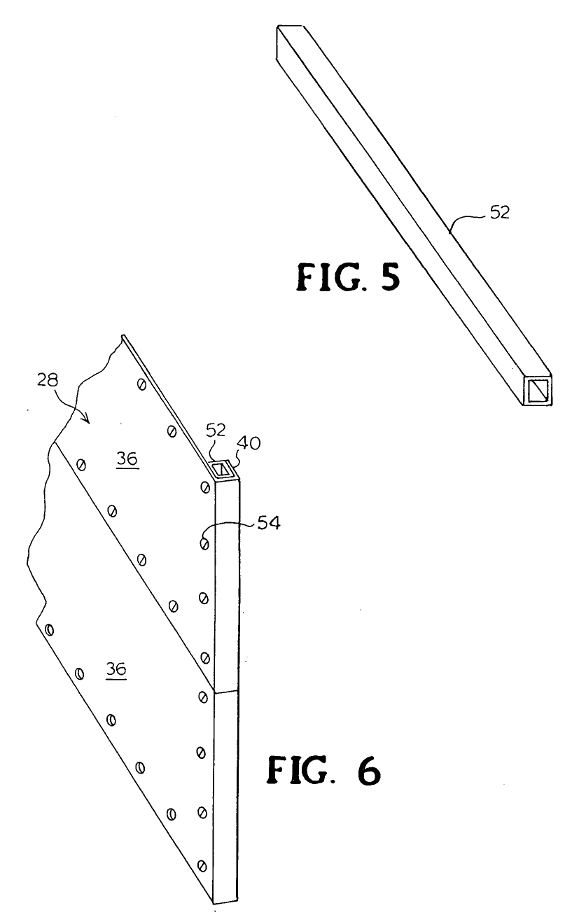

Four legs 52 (FIG. 5), made of 2×2 inch square 0.062 inch tubing, 83.87 inches long for the standard apparatus 20, or other length so that they fit along each corner of the selected apparatus size, are positioned one at each corner of the apparatus 20 as shown in FIG. 6. The legs are preferably made of 6063-T6 aluminum, or alternatively, may be made of stainless steel.

In assembly of the apparatus 20, the U-flanges 40 of the sides are placed around the respective legs 52 as shown (FIG. 6). The sides 28 are preferably attached to the legs 52 by self-drilling and tapping screws 54 (TREK screw) as are known in the art, in which case, holes 44 are not needed. Alternatively, each leg 52 may have holes 44 along the sides of the square tube and the U-flanges may have corresponding holes to bolt them to the legs. Each leg preferably sits in one corner of pan 46.

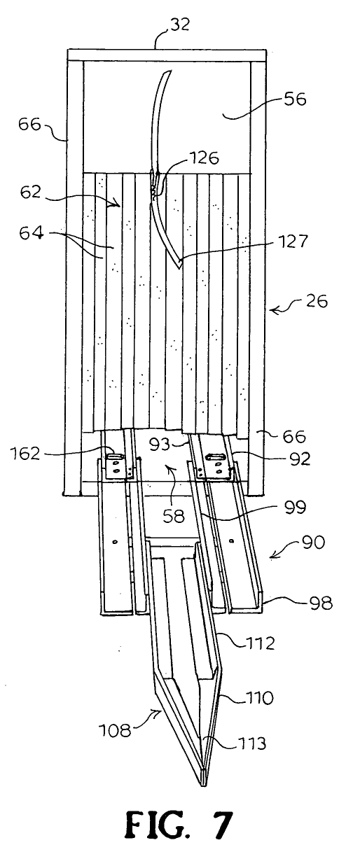

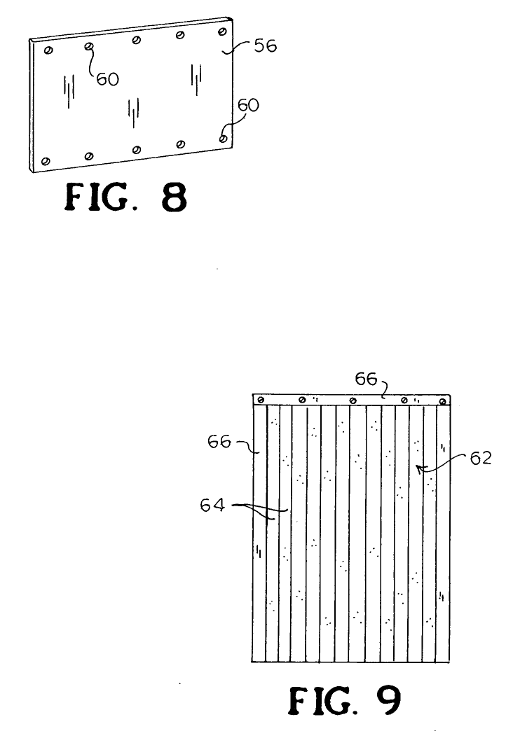

Both the first entry end 26 and second exit end 30 have an upper panel 56 (FIG. 8) over an opening 58 through which the carts 22 may be moved (FIG. 7). Each upper panel 56 is preferably 19 inches high by 35.87 inches high, and is formed of stainless steel. There are holes 60 (e.g., 0.187 inch diameter) at equal intervals (e.g., 5-inch intervals) along the top and bottom of each upper panel 56. With this size upper panel 56, an opening 58 of 61×36 inches is left below the panel 56 on each end in the preferred embodiment.

A sheet 62 of all-weather carpet, 60 inches high×36 inches wide, cut from the bottom into 4 inch strips 64 hanging from the top (FIGS. 7 and 9), is mounted on each end of the apparatus 20 beneath upper panel 56 by means of bolts through metal slats 66 so that the strips 64 cover the opening to reduce splashing of water to the outside of the apparatus 20 during washing, but allow the carts 22 to be moved through the opening 58 into and out of the apparatus 20. One or more central strip(s) may be removed if desired (not shown) to make it easier to move the carts 22 through the apparatus 20.

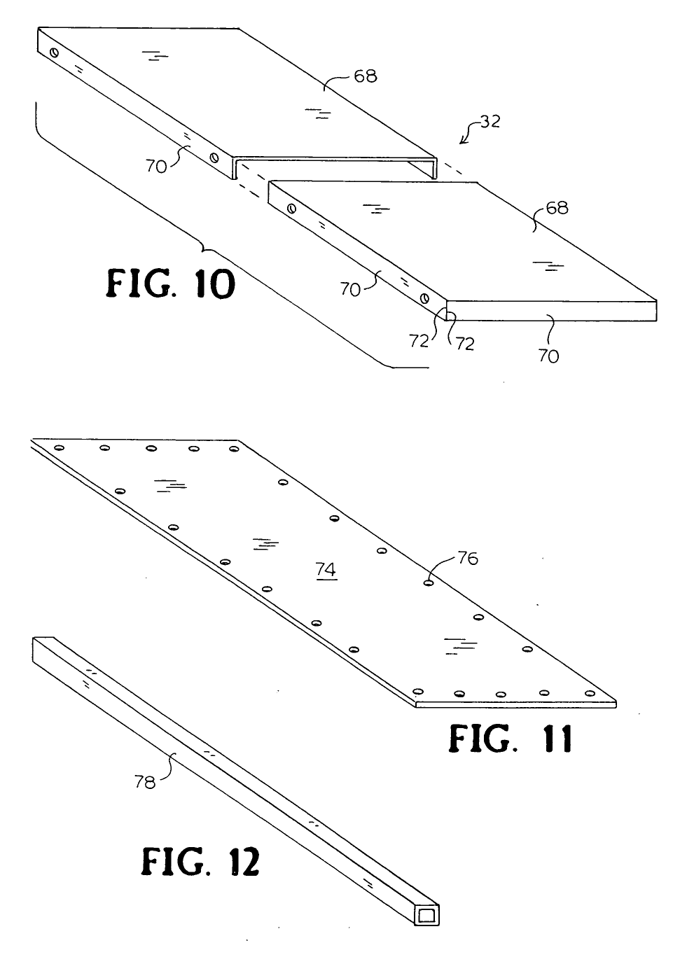

Two cover pieces 68 (FIG. 10), each made of either soft aluminum (0.063 inches) or 26 gauge stainless steel, 45.62 inches×36.12 inches in size, are bent over to form 2-inch edges 70 welded together where their ends 72 meet. The edges 70 extend along the long sides and one of the short sides of each cover piece 68. Together the cover pieces 68 form the two-piece cover when placed on top of the apparatus 20 of the invention with the short sides that do not have edges meeting in the center of the apparatus 20.

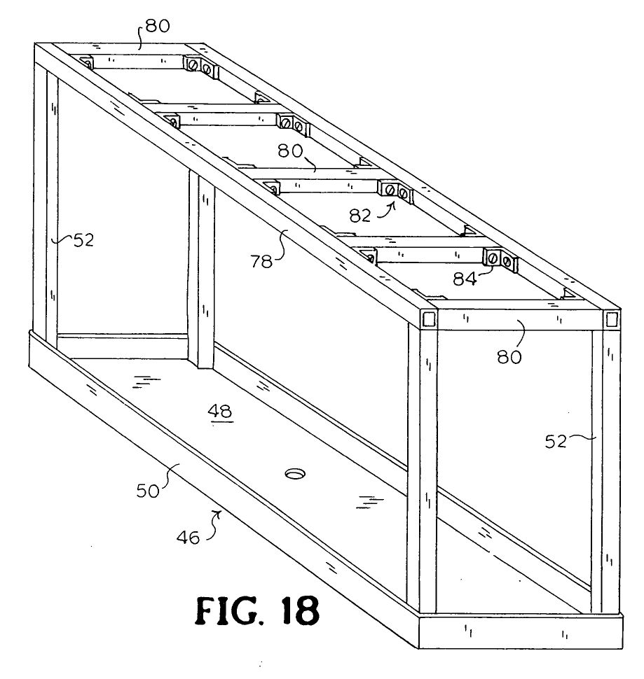

There are preferably four side rails 78 (two upper and two lower), each 90.87 inches long in the preferred embodiment, and preferably made of 2×2×0.062 inch square tube (FIG. 12 shows one of the bottom side rails 78)). There may be 0.187 inch diameter holes in the upper side rails, along one side of each rail that is used at the top of the invention, one at each end of each rail, and six (6) more holes spaced in pairs along the rail, the members of each pair being 4 inches apart and each pair equally spaced from the next pair or from the end of the side rail (preferred hole locations are shown by the location of the screws on the upper side rail 78 in FIG. 18). Two of the side rails 78 are mounted along the long sides of the bottom of the apparatus 20, with the bottom of the long sides and attached side rails 78 preferably inside, or optionally, outside the pan itself. The upper two side rails 78 are bolted along the top side sheets 36 (FIG. 2).

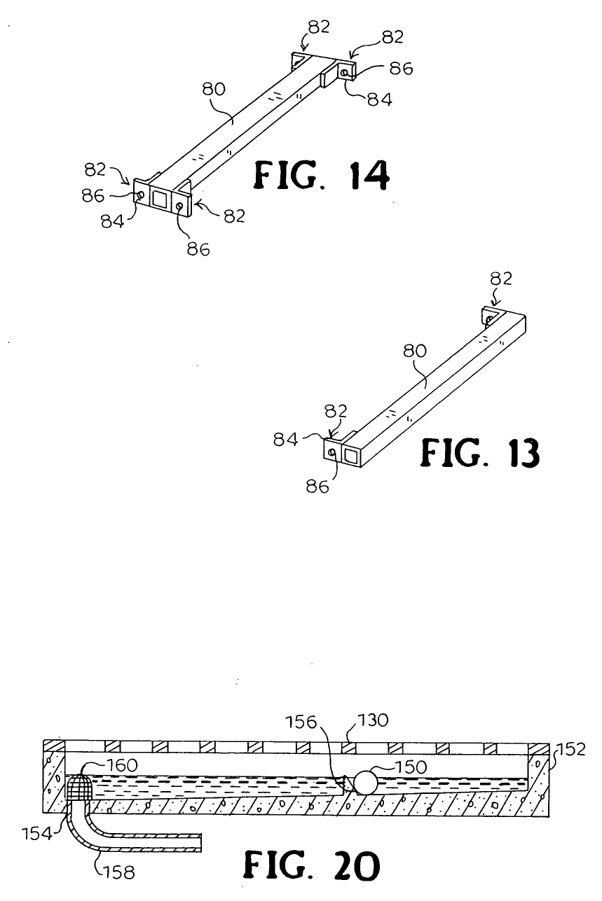

Equipment support brackets 80 (FIGS. 13-14) are mounted inside and at the top of the apparatus 20 extending across from one side to another between the upper side rails 78. Preferably there are five equipment support brackets 80. Each equipment support bracket 80 is preferably made of square 2×2×0.062 inch tube; however, 2.5×2.5×0.125 inch angle pieces may be used. There may be holes along the sides of the square tube as shown in FIG. 13 for bolting the two equipment support brackets that are at the two ends 26, 30 of the apparatus 20 to the upper panel 56. Alternatively these two equipment support brackets 80 may be attached to the upper panel 56 by means of self-drilling and tapping screws. There are optional holes in the top of the end equipment support brackets 80 for attachment of the top divider. The remaining fourteen equipment support brackets 80 (FIG. 14) do not have holes along their sides.

One or two L-shaped mounting brackets 82 are attached to each end of each equipment support bracket 80 (FIGS. 13-14) prior to attaching them to the apparatus 20. Each wing 84 of each L-shaped mounting bracket 82 is preferably 1.5×2.5 inches and has a centered 0.25-inch diameter hole 86 as shown in FIGS. 13-14. One wing 84 of each L-shaped mounting bracket 82 is attached to an end of an equipment support bracket 82 by means of welding, with the other wing 84 of the L-shaped mounting bracket 82 extending at right angles to the linear axis of the support bracket 82. For the support bracket 82 at the front end 26 and the support bracket at the rear end 30 of the apparatus 20, there is only one L-shaped mounting bracket (FIG. 13). For the ends of the remaining support brackets 80, there are two attached L-shaped mounting brackets 82 as shown in FIG. 14. To assemble the support brackets in the apparatus 20, the support brackets are placed in position equally spaced across the interior of the apparatus 20, and bolted at each end to one of the upper side rails 78 using the L-shaped mounting brackets 82.

A top divider 74 of the structure is preferably 35.75 inches wide, 90.75 inches long and 0.025 inches thick (FIG. 11). Holes 76 spaced at 10-inch intervals are positioned along each side 1.5 inches from the side of the top divider 74, and are used to attach the top divider 74 to the tops of the side rails 78 that are at the top of the assembled sides.

A tank 124 (FIG. 2), preferably a rectangular 75-gallon tank is mounted on top of the divider 74 toward the first entry end 26, above the interior enclosure in the apparatus 20. The preferred tank 124 is preferably made of low-density polyethylene designed for commercial containment of liquids and for level viewing.

The invention includes a mechanism for automatic triggering of release of water from the tank 124, which comprises: a snap-action switch 126 (FIG. 7) as known in the art. Because the trigger mechanism of these switches is generally quite short, a piece of tubing 127 is preferably placed over the trigger mechanism as shown in FIG. 7 to extend it so that when a cart 22 is pushed into the opening at the first entry end of the apparatus 20, the cart 22 hits the tubing causing the inserted trigger mechanism to move and trigger operation of the apparatus 20. Thus when one or more carts 22 are pushed/pulled into the first entry end 26, power relay is turned on, which causes the AC water pumps to be switched on to deliver water to the sprayers, and the blower 142 to start (see below).

The apparatus 20 of the invention preferably has two electrical control systems, an AC system for the dry components and a DC system for the components exposed to liquid, assembled as is known in the art for accomplishing the functions of the invention herein, the parts of which are shown schematically in FIG. 2. In the preferred embodiment of the invention, the AC equipment mounted on the top divider 74 toward the exit end 30, preferably comprises components as known in the art (FIG. 2), including a blower 142, pumps 144, one of which pumps water to upper sprayers 128 and one of which pumps water to the lower sprayers 132.

Other AC components (shown schematically and grouped together as 140 in FIG. 2), including a delay solid state timer (e.g., 120 V AC, 0.5 minute delay) for keeping the unit running for a selected period of time after the switch is turned off, so that the carts 22 are pulled entirely through the apparatus 20 when cleaned, a heavy-duty power relay operated by the microswitch which turns on the two pumps, a main disconnect for AC power (30 amp), a float switch for turning on the pump to pump clean liquid to fill the tank, and a control box. These components are used to control the sprayer pumps, the battery charger, and the furnace blowers as is known in the art.

The DC equipment comprises a battery charger 146 and a battery 148 (FIG. 2), a sump pump 150 (FIG. 20) for pumping out the excess used sanitizing fluid, either to a drain system or tank, or elsewhere outside the apparatus 20 as is appropriate for the location of use, and the winch 116 (FIGS. 1a and 1b)(discussed below).

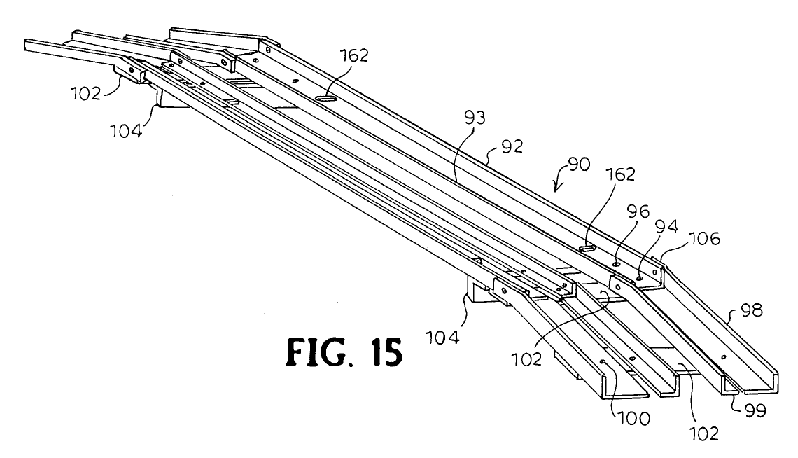

A series of guide rails 90 formed into a rail system allows carts 22 to be pushed or pulled through the apparatus 20 the invention, preferably with the winch 116 as discussed herein. In this embodiment, preferably there are six rail system components as shown in FIG. 15, including: 1) two 111-inch long extended outside angle pieces 92 (2.5×10.0×⅛ inch) preferably made of aluminum, having a first hole 94 that is five inches from each end and a second hole 96 that is 12 inches from each end, and which extend through the apparatus 20 in the preferred embodiment of the invention; 2) two 111-inch long extended inside angle pieces 93 (2.5×2.5×⅛ inch) preferably made of aluminum, having a first hole 94 that is five inches from each end and a second hole 96 that is 12 inches from each end, and which extend through the apparatus 20 in the preferred embodiment of the invention; 3) four (two for a ramp at each end) 36-inch long shorter outside angle pieces 98 (2.5×10.0×⅛ inch) having a hole 100 that is five inches from one end; 4) four (two for a ramp at each end) 36-inch long shorter inside angle pieces 99 (2.5×2.5×⅛ inch) having a hole 100 that is five inches from one end; 5) two cross-wise support bars 102 for each end of the rail system, having dimensions of 29×3 inches, and having four 0.34-inch diameter holes (not shown) spaced at 7.6 inch intervals so that the ends of the extended angle pieces 92, 93 may each be attached to a hole in the cross-wise support bar 102; and 6) a cross-wise angled piece 104 having four holes (not shown) on one face at the same spacing as for the cross-wise support bar which is attached to the extended angle pieces 92,93 at the second hole 96 thereof. In addition, there may be corner angled (15 degrees) mounts for each end of the apparatus 20 for attaching the extended angle pieces 92,93 to the shorter angle pieces 98,99 to form an angled ramp at each end as is known in the art (not shown). The spacing of the four parallel angle pieces 92,93 is designed so that the more closely spaced front wheels of standard shopping carts 22 (about 7.6 inches apart) can ride on the two interior angle pieces 93, and the more widely spaced rear wheels (about 24 inches apart) can ride on the two exterior angle pieces 92. Thus, for carts having different wheel spacings, the dimensions and spacings of the rail system would be appropriately altered.

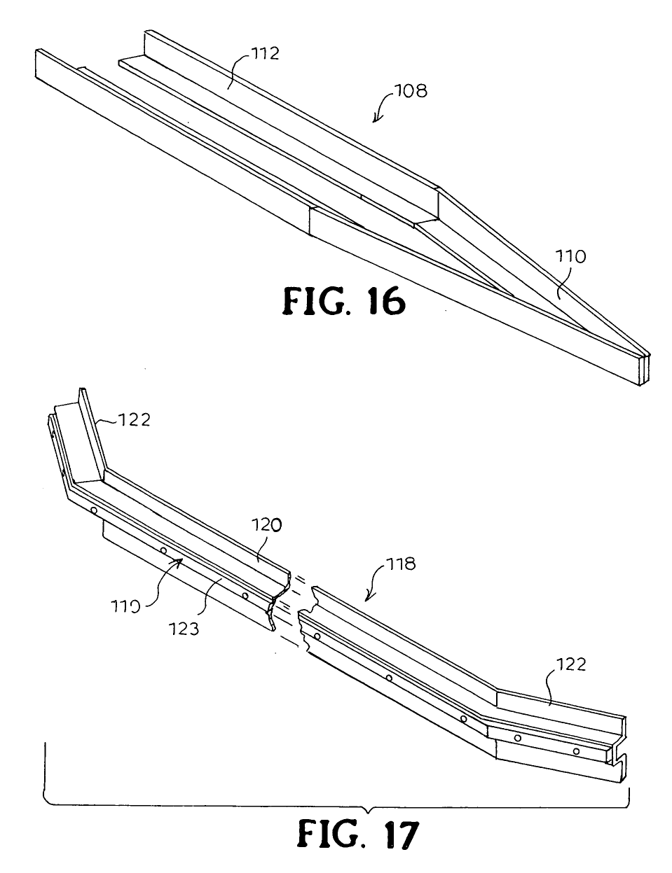

A separate external triangular guide rail 108 (FIG. 16) is preferably provided to enable the wheels of the carts 22 to be easily aligned when carts are being positioned for entry into the apparatus 20 of the invention, particularly in the embodiment in which the winch is used to pull the carts into the apparatus 20. The triangular guide rail 108 is preferably made of two short (24-inch) 3×3×¼ angle steel pieces 110 and two long (36-inch) 3×3×¼ angle steel pieces 112. A first end of each short angle steel piece 110 is welded to a first end of a long angle steel piece 112, and the second end of the two short angle pieces 110 is welded together as shown in FIG. 16 so that the distance between the free ends of the long angle steel pieces 112 is slightly less than the distance between the front wheels of the shopping carts 22 (7 inches in the example herein) and the vertical portion of the angled steel piece is on the outside edge of the triangular guide rail. At the outer point where the two short steel pieces 110 are joined, the horizontal portion of each is cut off at an angle 113 as shown in FIG. 7, so that the steel pieces can be joined and welded together to lie flat on the ground surface. Painting the external triangular guide rail a bright color such as yellow makes it easier to align the triangular guide rail with the rail system and to align the carts 22 with the triangular guide rail.

The carts 22 may be manually pushed through the apparatus 20, but preferably the rearmost cart 22 of a nested group of carts is attached to a hook as is known in the art (not shown) that is at the end of a long (e.g., 35 feet) belt 114 that is attached to a winch 116 as known in the art, preferably mounted on the top divider 74 of apparatus 20 at the exit end 30. To use the winch, the belt from the winch is pulled through the apparatus 20 from its mounted location at the exit end 30, through the apparatus 20 and out the entry end, to be hooked on to the cart(s). A safety cord (not shown) attached to the hook end and is located outside the apparatus 20 so that the hook may be positioned as desired without the operator needing to go inside the apparatus 20 (as known in the art; not shown) enables the operator of the apparatus 20 to retrieve the hook end and move it as desired. The preferred winch 116 has the following characteristics: 1500 lb. pull, 2-2.5 inch belt, 2000 lb. capacity, pull rate of 6.9 feet/minute. As noted, a belt 114, rather than a cable, is preferably used with the winch 116, to optimize rolling up of the belt when drawn in by the winch.

In an alternative embodiment, in which carts 22 are manually pushed through the apparatus 20 of the invention and no winch is used, there need not be rails as described above. In this embodiment shown generally in FIG. 1A), a hat section 118 (FIG. 17) is attached inside each side (not shown attached) along the area where the two sheets 36 overlap, by means of bolts. The hat sections 118 serve as a guide so that carts 22 can funneled into the apparatus 20. Each hat section 118 comprises a length of metal having a central section 120 and two end sections 122 at an angle to the central section 120 as shown in FIG. 17. Along the entire length of metal there are two outer flanges for attachment to the inside of the side of the apparatus 20 and a central raised portion along which a flat nylon guide 123 is attached along the full length of the hat section. Preferably the flat nylon guide 123 serves as a rub rail for the carts or other items being sanitized and washed, and is made of white nylon to minimize marking of the carts or other items.

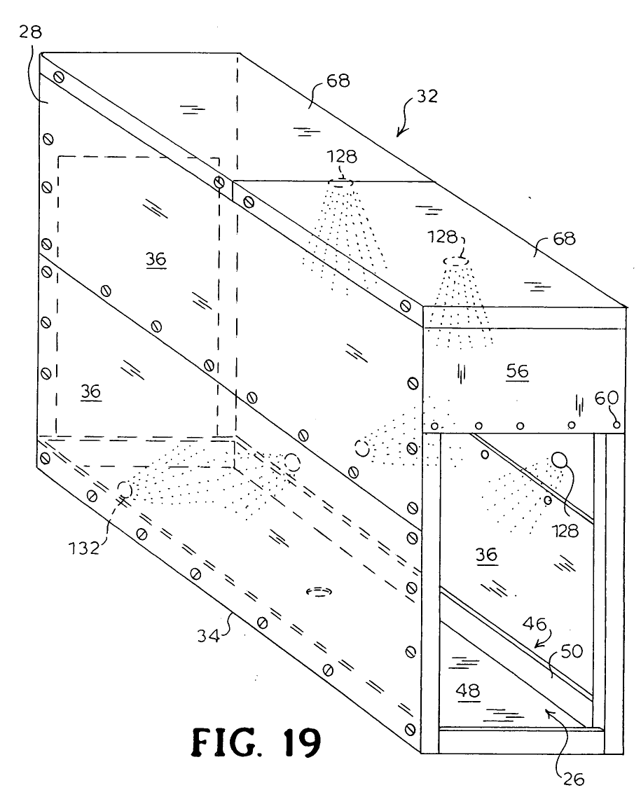

In use of the apparatus' sanitizer and washer system, water is conveyed from tank 124 to a series of upper sprayers 128, preferably four sprayers 128 positioned at the first end 26, with one sprayer 128 along each side of the apparatus 20, one sprayer 128 centrally placed near the first end at the top of the apparatus enclosure, and another sprayer 128 positioned at the top of the apparatus enclosure centrally between the sides of the enclosure and centrally between the ends of the enclosure (FIG. 19), preferably mounted underneath the top divider. Preferably the sprayer on each side is 32 inches from the bottom and 18 inches from the first end, which spray is directed at the handle and top of the cart 22. A second set of two sprayers 132 (one for each side) is located 30 inches from the first end, six inches from the bottom, for spraying at the bottom of the cart 22 and wheels (FIG. 19). From each sprayer 128,132, spray is released through a mister in an 8-inch thick stream, resulting in a 2×4 foot rectangular spray pattern where the stream hits the carts 22. The sprayers, for example upper sprayers 128, may have high pressure nozzles as is known in the art.

Two ½ HP portable self-priming centrifugal pumps 144, one for each side, preferably having a flow rate of 40-50 GPH at 52 psi/minute are used to control water flow to the sprayers. The Dayton 4CB57 (available from WW Grainger Equipment, which has a branch in Fort Worth, Tex.) is the preferred pump for this. Fluid is pumped from tank 124 to the upper sprayers 128, flows over the carts 22 during the sanitizing and washing and drains into pan 46. Used fluid is pumped with a second pump 144 to the lower sprayers 132 so that the lower portions of the carts 22 are washed, with used fluid draining back into pan 46. As fluid accumulates in pan 46, pump 150 (not shown, except in embodiment shown in FIG. 20 discussed below), having an autofloat switch as known in the art, pumps excess fluid out of pan 46.

In the version of the invention where the rails are not used, instead of having pan 46 at the bottom of the apparatus 20, there is a floor grating 130 over which the carts roll, made preferably of galvanized steel, aluminum or stainless steel (FIG. 1A). Thus a preferred grating is open steel floor grating, either a smooth-surface grating or a serrated-surface safety grating as preferred by the consumer, with standard or close mesh, for example, with mesh openings sufficient to allow drainage and not impede rolling of the carts, or as shown in FIG. 1A, the grating may simply comprise a series of parallel grate pieces perpendicular to the direction of travel of the carts (the arrow shown over the grating 130). Mesh examples include 15/16-1 3/16 inch×2-4 inches. Alternatively, an expanded flattened metal mesh as is known in the art may be used.

Beneath the grating in this version of the invention not having the guide rails is a drain pit 152 preferably made of concrete and having a slight downward slope to a lower drain 158, preferably from about 8 inches to about 10 inches deep (deeper at the drain end), with a central raised portion 156 to hold liquid from all immediately draining out of the drain pit 152 (FIG. 20). The drain pit 152 is preferably wide enough to fit under the entire apparatus 20 and extend beyond it to where the carts drain after being washed, for example, 4.0 feet in width, and preferably about 20 feet long. Fluid collected in the drain pit 152 exits and is conducted to the existing drain system by means of a 2-2.5 inch pipe at the apparatus location, e.g., into a sewer system. The pump 150 for recycling the chemical in this version is positioned in the drain pit. Preferably there is a shut off 160 having an automatic float switch as known in the art, so that when the liquid level is high, excess fluid is pumped out. There is preferably a screen at the drain pit exit 154 to keep large objects washed from the carts from getting into the drain system.

While the invention has been described with reference to specific embodiments, it will be appreciated that numerous variations, modifications, and embodiments are possible, and accordingly, all such variations, modifications, and embodiments are to be regarded as being within the spirit and scope of the invention.

What is claimed is:

1. A system for sanitizing articles, comprising:
a) a sanitizing solution capable of disinfecting the articles when sprayed on the articles without requiring rinsing of the articles after the articles have been sprayed with the sanitizing solution
b) a rectangular apparatus comprising a first entry end and a second exit end and defining an enclosure through which the articles may be transported, a tank for sanitizing solution, sprayers for spraying sanitizing solution on the top and bottom of articles in the enclosure, and a container for catching used sanitizing solution;
c) a mechanism for moving the articles through the rectangular enclosure from the first entry end and out of the enclosure at the second exit end;
d) a mechanism for releasing sanitizing solution from the tank when an article enters the enclosure;
e) a mechanism for carrying used sanitizing solution to the sprayers for spraying sanitizing solution on the bottom of the articles; and
f) a mechanism for carrying used sanitizing solution to a drain system.

2. The system for sanitizing articles according to claim 1, wherein the sanitizing solution comprises quaternary ammonium chloride.

3. The system for sanitizing articles according to claim 2, wherein the sanitizing solution comprises didecyl dimethyl ammonium chloride and n-alkyl dimethyl benzyl ammonium chloride.

4. The system for sanitizing articles according to claim 2, wherein the sanitizing solution comprises n-alkyl dimethyl benzyl ammonium chloride and n-alkyl dimethyl ethylbenzyl ammonium chloride.

5. The system for sanitizing articles according to claim 1, wherein the articles are shopping carts.

6. The system for sanitizing articles according to claim 5, further comprising guide rails for moving the shopping carts into and through the rectangular apparatus.

7. The system for sanitizing articles according to claim 1, further comprising strips of all-weather carpet placed at both the first entry end and at the second exit end.

8. The system for sanitizing articles according to claim 1, wherein the mechanism for releasing sanitizing solution comprises a snap-action switch which when triggered by an article at the first entry end causes the pumps to deliver water to the sprayers.

9. The system for sanitizing articles according to claim 8, further comprising a blower which is started when the snap-action switch is triggered.

10. The system for sanitizing articles according to claim 9, further comprising a delay solid state timer which keeps sanitizing solution flowing and the blower running for a selected amount of time when all articles have moved into the apparatus past the first entry end.

11. The system for sanitizing articles according to claim 1, wherein the mechanism for moving the articles through the rectangular enclosure comprises a winch connected to a hook that may be attached to the articles.

12. The system for sanitizing articles according to claim 1, wherein the container for catching used sanitizing solution is a pan beneath the rectangular apparatus.

13. The system for sanitizing articles according to claim 1, further comprising a floor grating, wherein the container for catching used sanitizing solution comprises a drain pit beneath the rectangular apparatus and outside the rectangular apparatus at the second exit end, wherein the floor grating extends over the drain pit.

14. The system for sanitizing articles according to claim 1, wherein the mechanism for moving articles through the rectangular apparatus comprises hat sections to guide the articles into the rectangular apparatus.

15. A method for sanitizing articles comprising:
a) providing: a system for sanitizing articles according to claim 1; sanitizing solution according to claim 1; and articles to be sanitized;
b) moving the articles to be sanitized into the rectangular enclosure from the first entry end;
c) spraying the articles to be sanitized with the sanitizing solution; and
d) moving the sanitized articles out of the enclosure at the second exit end.

16. The method for sanitizing articles according to claim 15, wherein the sanitizing solution comprises quaternary ammonium chloride.

17. The method for sanitizing articles according to claim 16, wherein the sanitizing solution comprises didecyl dimethyl ammonium chloride and n-alkyl dimethyl benzyl ammonium chloride.

18. The method for sanitizing articles according to claim 16, wherein the sanitizing solution comprises n-alkyl dimethyl benzyl ammonium chloride and n-alkyl dimethyl ethylbenzyl ammonium chloride.

19. The method for sanitizing articles according to claim 15, wherein the articles are shopping carts.
